(19) United States
(12) Patent Application Publication
(10) Pub. No.: US 2025/0346843 A1
(43) Pub. Date: Nov. 13, 2025

(54) DETECTION DEVICE

(71) Applicant: Japan Display Inc., Tokyo (JP)

(72) Inventors: Akihiko FUJISAWA, Tokyo (JP); Tomoyuki ISHIHARA, Tokyo (JP)

(21) Appl. No.: 19/194,887

(22) Filed: Apr. 30, 2025

(30) Foreign Application Priority Data
May 9, 2024 (JP) 2024-076523

(51) Int. Cl. C12M 1/34; C12M 1/22; C12M 3/00; H04N 23/667

(52) U.S. Cl. CPC C12M 1/3446; C12M 23/10; C12M 23/42; H04N 23/667

(57) ABSTRACT

According to an aspect, a detection device includes: a first optical sensor; a first light guide plate that is located on one side in a first direction with respect to the first optical sensor and has a light-transmitting property; an object placement member that is located on the one side in the first direction with respect to the first light guide plate and on which an object to be detected is to be placed; a second optical sensor located on the one side in the first direction with respect to the object placement member; and a first light source located adjacent to the first light guide plate in a second direction intersecting the first direction. A surface on another side of the first light guide plate is provided with first scatterers by which light propagating in the first light guide plate reaching thereto is scattered.

### CROSS-REFERENCE TO RELATED APPLICATION

This application claims the benefit of priority from Japanese Patent Application No. 2024-076523 filed on May 9, 2024, the entire contents of which are incorporated herein by reference.

### BACKGROUND

### 1. Technical Field

What is disclosed herein relates to a detection device.

### 2. Description of the Related Art

Japanese Patent Publication No. 6830593 (JP-6830593) discloses a biosensor that includes an optical sensor including a photosensor (photodetection element), a culture vessel placed on the upper side of an imaging surface of the photosensor, and a point light source disposed on the upper side of the culture vessel. The biosensor of JP-6830593 captures images in only one imaging mode in which light emitted from the point light source passes through a culture medium and enters the photosensor.

Detection devices with higher accuracy in detection of objects to be detected are required.

### SUMMARY

According to an aspect, a detection device includes: a first optical sensor including a plurality of photodetection elements arranged in a planar configuration; a first light guide plate that is located on one side in a first direction with respect to the first optical sensor and has a light-transmitting property; an object placement member that is located on the one side in the first direction with respect to the first light guide plate and on which an object to be detected is to be placed; a second optical sensor that is located on the one side in the first direction with respect to the object placement member and includes a plurality of photodetection elements arranged in a planar configuration; and a first light source that is located adjacent to the first light guide plate in a second direction intersecting the first direction and is configured to emit light to a side surface of the first light guide plate. A surface on another side in the first direction of the first light guide plate is provided with a plurality of first scatterers by which light propagating in the first light guide plate reaching thereto is scattered.

### BRIEF DESCRIPTION OF THE DRAWINGS

FIG. 1 is a schematic view illustrating a section of a detection device according to an embodiment of the present disclosure;

FIG. 2 is a block diagram illustrating a configuration example of the detection device according to the embodiment;

FIG. 3 illustrates schematic views illustrating operating states of the detection device according to first to fourth imaging modes;

FIG. 4 is a schematic view illustrating a state in which a mark is attached to a surface of a culture medium;

FIG. 5 is a flowchart illustrating an exemplary detection operation of the detection device to select and determine an optimal imaging mode using imaging data of the mark;

FIG. 6 illustrates schematic diagrams illustrating histograms of images of the mark;

FIG. 7 is a flowchart of a process to monitor bacterial colonies using the optimal imaging mode;

FIG. 8 is a schematic view illustrating a section of a detection device according to a modification of the embodiment;

FIG. 9 is a block diagram illustrating a configuration example of the detection device according to the modification; and

FIG. 10 is a flowchart illustrating an exemplary detection operation of the detection device to select and determine the optimal imaging mode using the imaging data of the mark.

### DETAILED DESCRIPTION

The following describes a mode (embodiment) for carrying out the present invention in detail with reference to the drawings. The present disclosure is not limited to the description of the embodiment given below. Components described below include those easily conceivable by those skilled in the art or those substantially identical thereto. In addition, the components described below can be combined as appropriate. What is disclosed herein is merely an example, and the present disclosure naturally encompasses appropriate modifications easily conceivable by those skilled in the art while maintaining the gist of the present disclosure.

To further clarify the description, the drawings may schematically illustrate, for example, widths, thicknesses, and shapes of various parts as compared with actual aspects thereof. However, they are merely examples, and interpretation of the present disclosure is not limited thereto. The same component as that described with reference to an already mentioned drawing is denoted by the same reference numeral through the present disclosure and the drawings, and detailed description thereof may not be repeated where appropriate.

In the drawings, a Z direction (first direction) corresponds to the up-down direction, and a Y direction (second direction) corresponds to the left-right direction. The Y direction intersects (at right angles) the Z direction. A Z1 side is one side in the first direction, and a Z2 side is the other side in the first direction. The Z2 side is the upper side, for example, and the Z1 side is the lower side, for example. A Y1 side is one side in the second direction, and a Y2 side is the other side in the second direction.

### Embodiment

An embodiment of the present disclosure will first be described.

### Configuration of Detection Device

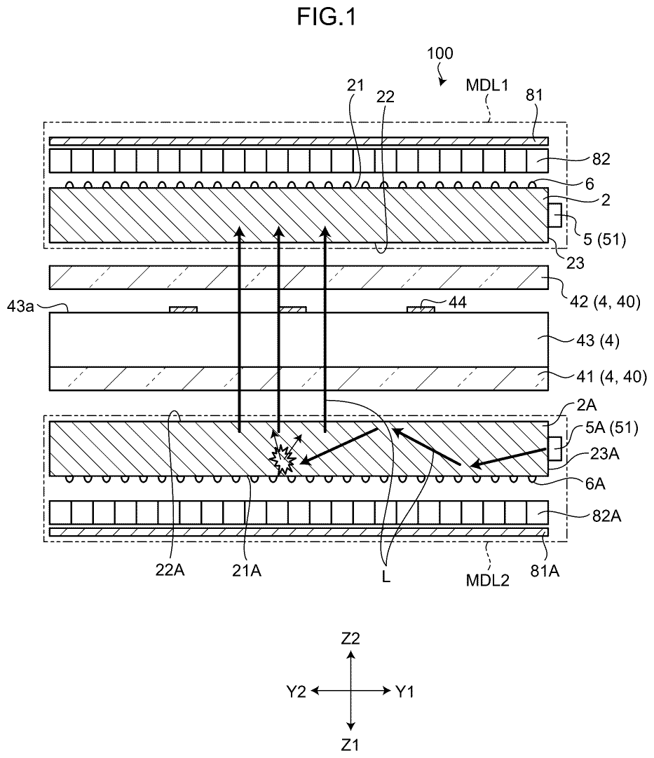

FIG. 1 is a schematic view illustrating a section of a detection device according to the embodiment. As illustrated in FIG. 1, a detection device 100 includes a first module MDL1, an object placement member 4, and a second module MDL2. The first module MDL1, the object placement member 4, and the second module MDL2 are arranged in this order from the Z2 side (other side in the first direction) to the Z1 side (one side in the first direction). That is, the object placement member 4 is placed on the Z1 side with respect to the first module MDL1, and the second module MDL2 is placed on the Z1 side with respect to the object placement member 4.

The first module MDL1 includes a first optical sensor 81, a first viewing angle control film 82, a first light guide plate 2, and first light sources 5. The first optical sensor 81, the first viewing angle control film 82, and the first light guide plate 2 are arranged in this order from the Z2 side toward the Z1 side.

The first optical sensor 81 will be described later. The first viewing angle control film 82 is an optical element that transmits, toward the first optical sensor 81, components of light L that travel in the Z direction. The first viewing angle control film 82 includes, for example, light-blocking portions and light guide portions. The light-blocking portions have higher light absorbance than the light guide portions. That is, the light L passing through the light guide portions exits toward the first optical sensor 81.

The first light guide plate 2 is a light-transmitting flat plate member. The first light guide plate 2 has a first surface 21, a second surface 22, and a side surface 23. The first surface 21 is a principal surface on the Z2 side, and the second surface 22 is a surface on the opposite side (that is, the Z1 side) to the first surface 21. The side surface 23 is located on the Y1 side. A plurality of first scatterers 6 are provided to be spaced on the first surface 21.

The first light sources 5 face the side surface 23 of the first light guide plate 2. The first light sources 5 are located on the Y1 side (one side in the second direction) of the side surface 23 of the first light guide plate 2. The first light sources 5 emit the light L to the side surface 23 of the first light guide plate 2. The first light sources 5 are a plurality of light-emitting diodes (LEDs) 51, for example. When the light L propagating in the first light guide plate 2 hits the first scatterers 6, the light L is scattered at the first scatterers 6.

The object placement member 4 includes, for example, a Petri dish 40 and a culture medium (agar) 43. The Petri dish 40 includes a bottom glass 41 and a cover glass 42. The bottom glass 41 is provided with the culture medium 43. Objects to be detected 44 are placed on a surface 43a of the culture medium 43. The objects to be detected 44 are, for example, microorganisms such as bacteria, or other micro-objects, such as cells.

The second module MDL2 includes a second optical sensor 81A, a second viewing angle control film 82A, a second light guide plate 2A, and second light sources 5A. The second optical sensor 81A, the second viewing angle control film 82A, and the second light guide plate 2A are arranged in this order from the Z1 side toward the Z2 side. The second optical sensor 81A, the second viewing angle control film 82A, and the second light guide plate 2A have the same structures as those of the first optical sensor 81, the first viewing angle control film 82, and the first light guide plate 2, respectively, described above. The second light sources 5A are the LEDs 51, for example. The second light sources 5A face a side surface 23A of the second light guide plate 2A on the Y1 side. A first surface 21A is a principal surface on the Z1 side, and a second surface 22A is a surface on the opposite side (that is, the Z2 side) to the first surface 21A. A plurality of second scatterers 6A are provided to be spaced on the first surface 21A.

As illustrated in FIG. 1, when the second light sources 5A are lit, the light L from the second light sources 5A first enters the inside of the second light guide plate 2A from the side surface 23A. Then, the light L is repeatedly reflected on the first surface 21A and the second surface 22A and travels toward the Y2 side, where the light L hits the second scatterers 6A and is scattered. By being scattered, the light L travels toward the Z2 side, passes through the object placement member 4, the first light guide plate 2, and the first viewing angle control film 82, and irradiates the first optical sensor 81.

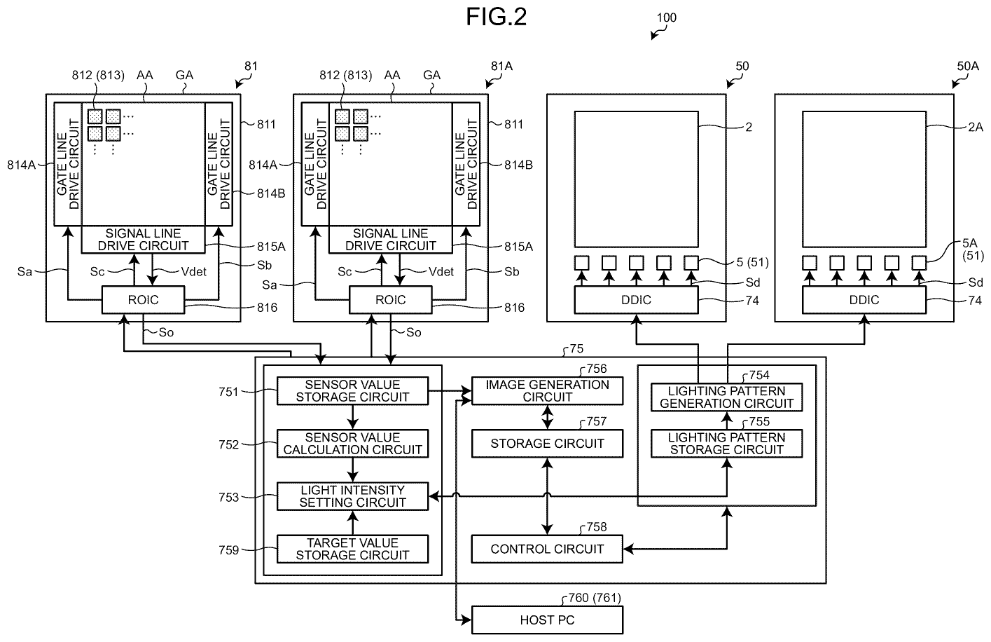

FIG. 2 is a block diagram illustrating a configuration example of the detection device according to the embodiment. As illustrated in FIG. 2, the detection device 100 includes the first optical sensor 81, the second optical sensor 81A, a first light source device 50, a second light source device 50A, a host integrated circuit (IC) 75, and a host personal computer (PC) 760.

The first optical sensor 81 and the second optical sensor 81A each include an array substrate 811, a plurality of sensor pixels 812 (photodiodes 813) formed on the array substrate 811, gate line drive circuits 814A and 814B, a signal line drive circuit 815A, and a detection control circuit (ROIC) 816. Photodetection elements are the sensor pixels 812 or the photodiodes 813.

The array substrate 811 is formed using a substrate as a base. Each of the sensor pixels 812 is configured with a corresponding one of the photodiodes 813, a plurality of transistors, and various types of wiring.

The array substrate 811 has a detection area AA and a peripheral area GA. The detection area AA is an area provided with the sensor pixels 812 (photodiodes 813). The peripheral area GA is an area between the outer perimeter of the detection area AA and the outer edges of the array substrate 811, and is an area not provided with the sensor pixels 812. The gate line drive circuits 814A and 814B, the signal line drive circuit 815A, and the detection control circuit 816 are provided in the peripheral area GA.

Each of the sensor pixels 812 is an optical sensor that includes the photodiode 813 as a sensor element. Each of the photodiodes 813 outputs an electrical signal corresponding to light emitted thereto.

The detection control circuit 816 is a circuit that supplies control signals Sa, Sb, and Sc to the gate line drive circuits 814A and 814B and the signal line drive circuit 815A, respectively, to control operations of these circuits. The detection control circuit 816 includes a signal processing circuit that processes a detection signal Vdet output from each of the photodiodes 813.

The detection control circuit 816 processes the detection signal Vdet output from the photodiode 813 and outputs, to the host IC 75, a sensor value So based on the detection signal Vdet.

The first light source device 50 includes the first light sources 5 (LEDs 51) and a light-emitting element control circuit (DDIC) 74. The first light sources 5 (LEDS 51) are located so as to face the side surface 23 of the first light guide plate 2. The first light sources 5 are driven to be switched between on (lit state) and off (unlit state) by a command Sd of the light-emitting element control circuit 74. The second light source device 50A includes the second light sources 5A (LEDs 51) and the light-emitting element control circuit 74, and the second light sources 5A (LEDs 51) are located so as to face the side surface 23A of the second light guide plate 2A.

The host IC 75 functions as a control circuit for the first optical sensor 81 and the second optical sensor 81A and includes a sensor value storage circuit 751, a sensor value calculation circuit 752, a light intensity setting circuit 753, a target value storage circuit 759, and a storage circuit 757. The sensor value storage circuit 751 stores therein the sensor values So output from the detection control circuits 816 of the first optical sensor 81 and the second optical sensor 81A. The sensor value calculation circuit 752 performs a predetermined calculation process on the sensor values So of the photodiodes 813.

In a light intensity setting mode, the light intensity setting circuit 753 sets light intensities of the first light sources 5 and the second light sources 5A for detection by comparing the sensor values So detected by the photodiodes 813 with a preset target sensor value So-t acquired from the target value storage circuit 759. The target value storage circuit 759 stores therein the preset target sensor value So-t.

The host IC 75 includes a lighting pattern generation circuit 754 and a lighting pattern storage circuit 755. The lighting pattern storage circuit 755 stores therein information on the light intensity of each of the first and the second light sources 5 and 5A in the light intensity setting mode.

The lighting pattern generation circuit 754 generates various control signals based on the information on the light intensity in the lighting pattern storage circuit 755.

In a detection mode, an image generation circuit 756 generates various images based on the sensor values So output from the photodiodes 813.

The host IC 75 further includes the storage circuit 757 and a control circuit 758. The storage circuit 757 stores therein a base image. The base image is, for example, an image obtained by detecting the light L emitted from the first light sources 5 or the second light sources 5A using the first optical sensor 81 or the second optical sensor 81A while the objects to be detected 44 are not placed on the object placement member 4. The base image is stored at the time of designing or shipping the detection device 100. The base image may be, for example, a base image detected by placing a black board instead of the object placement member 4. The control circuit 758 is a controller that determines the imaging mode illustrated in TABLE 1 to be described later with reference to table data illustrated in TABLE 2. TABLES 1 and 2, the imaging modes, and the table data will be described later.

The host PC 760 receives images generated by the image generation circuit 756 and stores therein the images. The host PC 760 includes an input device 761. The input device 761 receives the input of specified combinations of conditions in the table data illustrated in TABLE 2. The storage circuit 757 includes a storage, which stores the table data (refer to TABLE 2) indicating a relation between a combination of two or more conditions and the imaging modes.

### Imaging Modes

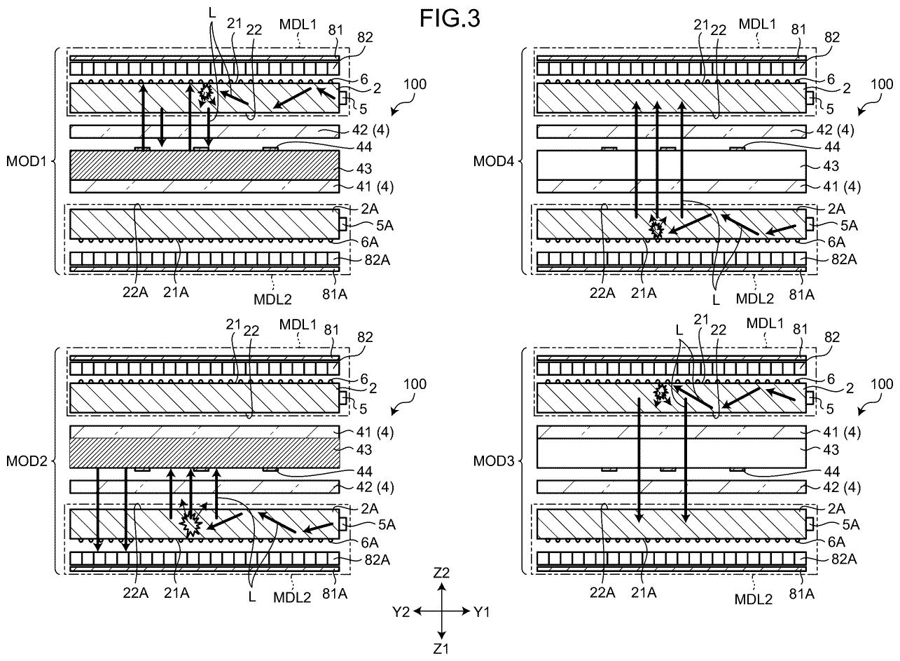

FIG. 3 illustrates schematic views illustrating operating states of the detection device according to first to fourth imaging modes. TABLE 1 below is a table illustrating content of the first to the fourth imaging modes.

TABLE 1

| IMAGING MODE | MDL1 | MDL2 |
|---|---|---|
| MOD1 (REFLECTION) | FL TYPE SENSOR | NOT USED |
| MOD2 (REFLECTION) | NOT USED | FL TYPE SENSOR |
| MOD3 (TRANSMISSION) | LIGHT SOURCES | SENSOR |
| MOD4 (TRANSMISSION) | SENSOR | LIGHT SOURCES |

As illustrated in FIG. 3 and TABLE 1, four imaging modes are provided according to the embodiment. The following describes a first imaging mode MOD1 to a fourth imaging mode MOD4.

In the first imaging mode MOD1, the first module MDL1 is operated and the second module MDL2 is not operated. Specifically, when the first light sources 5 in the first module MDL1 are lit, the light L propagating in the first light guide plate 2 hits the first scatterers 6, is scattered, and travels toward the Z1 side. The light L is then reflected on the culture medium 43, passes through the first light guide plate 2 and the first viewing angle control film 82, and irradiates the photodiodes 813 of the first optical sensor 81.

In the second imaging mode MOD2, the second module MDL2 is operated and the first module MDL1 is not operated. Specifically, when the second light sources 5A in the second module MDL2 are lit, the light L propagating in the second light guide plate 2A hits the second scatterers 6A, is scattered, and travels toward the Z2 side. The light L is then reflected on the culture medium 43, passes through the second light guide plate 2A and the second viewing angle control film 82A, and irradiates the photodiodes 813 of the second optical sensor 81A.

In the third imaging mode MOD3, the first module MDL1 and the second module MDL2 are operated. Specifically, when the first light sources 5 in the first module MDL1 are lit, the light L propagating in the first light guide plate 2 hits the first scatterers 6, is scattered, and travels toward the Z1 side. The light L then passes through the culture medium 43, the second light guide plate 2A, and the second viewing angle control film 82A, and irradiates the photodiodes 813 of the second optical sensor 81A.

In the fourth imaging mode MOD4, the first module MDL1 and the second module MDL2 are operated. Specifically, when the second light sources 5A in the second module MDL2 are lit, the light L propagating in the second light guide plate 2A hits the second scatterers 6A, is scattered, and travels toward the Z2 side. The light L then passes through the culture medium 43, the first light guide plate 2, and the first viewing angle control film 82, and irradiates the photodiodes 813 of the first optical sensor 81.

Methods for Selecting and Determining Imaging Mode Two methods can be used to determine which of the above-described four imaging modes is to be selected. The following describes first and second methods for selecting and determining the imaging mode.

### First Method for Selection and Determination

First, in the first method for selection and determination, an imaging mode corresponding to a combination of two or more conditions is determined with reference to the table data. TABLE 2 below illustrates an example of the table data. The two or more conditions include a condition regarding the transmittance of the culture medium 43 and the orientation of the surface 43a of the culture medium 43. The condition regarding the transmittance of the culture medium includes at least one of the type of the Petri dish 40, the thickness of the culture medium 43, and the transmittance of the culture medium 43.

TABLE 2 is the table data indicating a correspondence relation between: the transmittance of the culture medium 43 and the orientation of the surface 43a of the culture medium 43; and the imaging mode. The transmittance of the culture medium 43 is the transmittance when the highest transmittance is assumed to be 100 and the lowest transmittance is assumed to be 0. “Up” illustrated in TABLE 2 indicates a state where that the surface 43a of the culture medium 43 faces the Z2 side, and “Down” indicates a state where the surface 43a of the culture medium 43 faces the Z1 side.

TABLE 2

| | CULTURE MEDIUM | |
|---|---|---|
| TRANSMITTANCE | SURFACE | IMAGING MODE |
| ≥50 | Up | 4 |
| | Down | 3 |
| <50 | Up | 1 |
| | Down | 2 |

For example, if the transmittance of the culture medium 43 is lower than 50 and the surface 43a of the culture medium 43 faces the Z2 side, the first imaging mode MOD1 is selected. If the transmittance of the culture medium 43 is equal to or higher than 50 and the surface 43a of the culture medium 43 faces the Z1 side, the third imaging mode MOD3 is selected. Thus, the table data indicates the relation between the combination of the two or more conditions and the imaging mode, where the conditions are the transmittance of the culture medium 43, the orientation of the surface 43a of the culture medium 43, and the like.

### Second Method for Selection and Determination

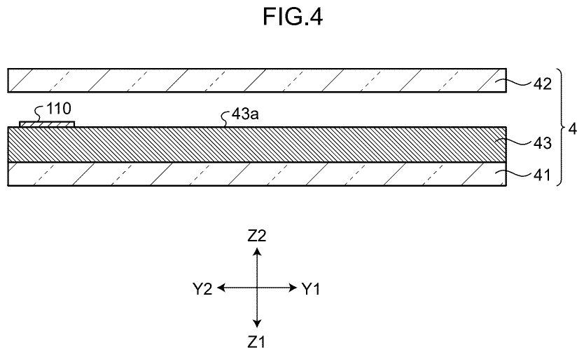

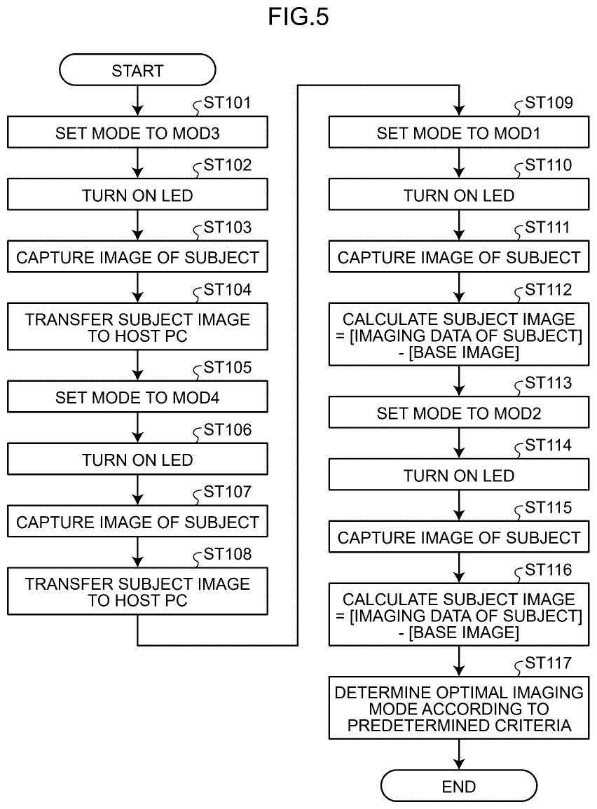

FIG. 4 is a schematic view illustrating a state in which a mark is attached to a surface of the culture medium. FIG. 5 is a flowchart illustrating an exemplary detection operation of the detection device to select and determine an optimal imaging mode using imaging data of the mark.

In the second method for selection and determination, a mark 110 is attached to a corner of the surface 43a of the culture medium 43 as illustrated in FIG. 4, the mark 110 is imaged (image-captured) in each of the four imaging modes, and the imaging mode that produces the clearest image of the mark is determined. The term “mark” is also referred to as a “resolution chart” or a “fine pattern”. That is, the object placement member 4 includes the Petri dish 40 and the culture medium 43 accommodated in the Petri dish 40, and the mark 110 is provided in advance on (a part of) the surface 43a of the culture medium 43. The following briefly describes the second method for selection and determination with reference to the flowchart in FIG. 5.

In FIG. 5, the mark 110 is imaged in the order of the third imaging mode MOD3, the fourth imaging mode MOD4, the first imaging mode MOD1, and the second imaging mode MOD2. Then, the imaging mode that produces the clearest image of the mark is determined. A detailed description will be made below.

First, the host IC 75 sets the imaging mode to the third imaging mode MOD3 described with reference to FIG. 3 (Step ST101). That is, the lighting pattern generation circuit 754 turns on the first light sources 5 (Step ST102) and images (captures an image of) the mark 110 serving as a subject (Step ST103). The image generation circuit 756 then generates an image of the mark 110. The image generation circuit 756 transfers the image to the host PC 760, and the host PC 760 stores therein the image (Step ST104).

Then, the host IC 75 sets the imaging mode to the fourth imaging mode MOD4 (Step ST105). That is, the lighting pattern generation circuit 754 turns on the second light sources 5A (Step ST106) and images (captures an image of) the mark 110 serving as the subject (Step ST107). The image generation circuit 756 then generates an image of the mark 110. The image generation circuit 756 transfers the image to the host PC 760, and the host PC 760 stores therein the image (Step ST108).

Then, the host IC 75 sets the imaging mode to the first imaging mode MOD1 (Step ST109). That is, the lighting pattern generation circuit 754 turns on the first light sources 5 (Step ST110) and images (captures an image of) the mark 110 serving as the subject (Step ST111). The image generation circuit 756 then generates an image of the mark 110.

In detail, the base image is stored in advance in the storage circuit 757, as described above. The image generation circuit 756 performs an operation to subtract the base image from the imaging data of the mark 110 to obtain the image of the mark 110, and transfers the image of the mark 110 to the host PC 760. The host PC 760 stores therein the image (Step ST112).

Then, the host IC 75 sets the imaging mode to the second imaging mode MOD2 (Step ST113). That is, the lighting pattern generation circuit 754 turns on the second light sources 5A (Step ST114) and images (captures an image of) the mark 110 serving as the subject (Step ST115). The image generation circuit 756 then generates an image of the mark 110.

In detail, the base image is stored in advance in the generation circuit 756 performs an operation to subtract the base image from the imaging data of the mark 110 to obtain the image of the mark 110, and transfers the image of the mark 110 to the host PC 760. The host PC 760 stores therein the image (Step ST116).

Then, at Step ST117, the optimal imaging mode is determined according to predetermined criteria.

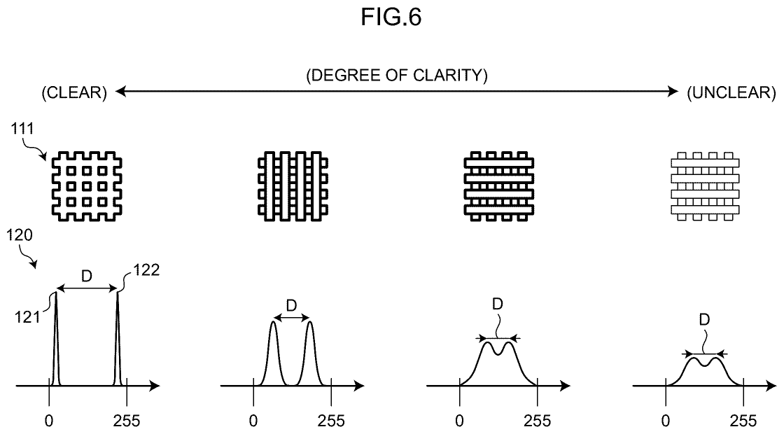

The following describes the detailed content of the process at Step ST117. FIG. 6 illustrates schematic diagrams illustrating histograms of the images of the mark. Four images of the mark 110 obtained from four times of the imaging processing in FIG. 5 are, for example, images 111 of the mark illustrated in the upper row of FIG. 6. The degree of clarity becomes higher from the right side to the left side of FIG. 6. The lower row of FIG. 6 illustrates the histograms (gradations representing color densities) corresponding to the images 111 illustrated in the upper row. In the histograms, the brightness is indicated as the class on the horizontal axis and the number of pixels (points) of the class is stacked on the vertical axis. The difference in gradation between a peak 121 on the black side and a peak 122 on the white side is indicated by a difference D between peak gradations (hereinafter, referred to as a peak gradation difference D). The peak gradation difference D increases toward the left side in FIG. 6. That is, the peak gradation difference D decreases as the image blurs more.

Therefore, of the four images of the mark 110, a mode that produces an image that has the largest peak gradation difference D is determined by the host IC 75 as the optimal imaging mode.

### Detection of Bacterial Colonies

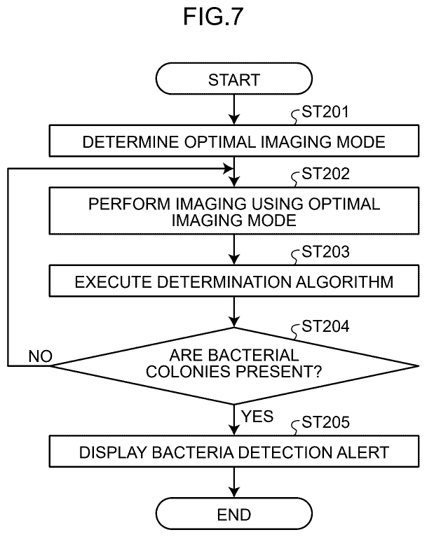

Then, the culture medium is imaged using the optimal imaging mode selected according to the flowchart in FIG. 5 and the histogram of the image of the mark in FIG. 6, and a development of the microorganisms such as bacterial colonies (objects to be detected) is detected. FIG. 7 is a flowchart of a process to monitor the bacterial colonies using the optimal imaging mode.

As illustrated in FIG. 7, first, at Step ST201, the optimal imaging mode is determined as described above. Then, at Step ST202, the culture medium 43 is imaged using the optimal imaging mode. At Step ST203, a determination algorithm is used to determine whether the bacterial colonies are present on the surface 43a of the culture medium 43. If the bacterial colonies are not present (No, at Step ST204), the process at Step ST202 is performed. If the bacterial colonies are present (Yes, at Step ST204), a process at Step ST205 is performed. At Step ST205, an alert that the bacteria have been detected is displayed on a user interface or the like. Thus, with the operation illustrated in the flowchart in FIG. 7, the Petri dish possibly having bacteria applied thereon can be monitored on a time-series basis, whereby the bacterial colonies can be detected early.

As described above, the detection device 100 according to the embodiment includes the first optical sensor 81, the first light guide plate 2, the object placement member 4, the second light guide plate 2A, the second optical sensor 81A, the first light sources 5, the second light sources 5A, the first scatterers 6, and the second scatterers 6A.

As described above, the biosensor of JP-6830593 captures images in only one imaging mode in which the light emitted from the point light source passes through the culture medium in the culture vessel and enters the photosensor.

In contrast to this, the detection device 100 according to the embodiment can image (that is, capture the image of) the object placement member 4 and the objects to be detected 44 using the plurality of imaging modes. That is, an imaging mode that can produce the clearest image is selected from among the plurality of imaging modes, and the objects to be detected 44 and the object placement member 4 can be imaged in the selected imaging mode. Thus, the detection device 100 that improves the accuracy of detection can be provided according to the present embodiment. When the object placement member 4 includes the culture medium 43 and microorganisms (objects to be detected 44) are applied to the surface 43a of the culture medium 43, the development of the microorganisms (objects to be detected 44) applied to the culture medium 43 can be detected earlier by imaging the culture medium 43 in the imaging mode in which the clearest image is obtained.

The four imaging modes include the first imaging mode MOD1, the second imaging mode MOD2, the third imaging mode MOD3, and the fourth imaging mode MOD4. In the first imaging mode MOD1, the first light sources 5 are lit, and the light thereof is detected by the first optical sensor 81. In the second imaging mode MOD2, the second light sources 5A are lit, and the light thereof is detected by the second optical sensor 81A. In the third imaging mode MOD3, the first light sources 5 are lit, and the light thereof is detected by the second optical sensor 81A. In the fourth imaging mode MOD4, the second light sources 5A are lit, and the light thereof is detected by the first optical sensor 81.

Specifically, since the detection device 100 can image (that is, capture the image of) the object placement member 4 and the objects to be detected 44 using the four imaging modes, the objects to be detected 44 and the object placement member 4 can be imaged in the imaging mode that produces the clearest image among the four imaging modes.

The detection device 100 includes the storage (storage circuit 757) that stores therein the table data, the input device 761 that receives the input of the specified combination of the two or more conditions, and the controller (control circuit 758) that determines, with reference to the table data, the imaging mode corresponding to the combination of the conditions specified by the received input. The two or more conditions include the condition regarding the transmittance of the culture medium 43 and the orientation of the surface 43a of the culture medium 43.

This configuration is convenient because the optimal imaging mode in which the clearest image can be captured is automatically determined by simply inputting the specified combination of the two or more conditions.

The condition regarding the transmittance of the culture medium includes at least one of the type of the Petri dish 40, the thickness of the culture medium 43, and the transmittance of the culture medium 43.

This configuration allows the optimal imaging mode to be determined by a simpler operation because the number of conditions to be specified and to be input is smaller.

The mark 110 is provided in advance on (a part of) the surface 43a of the culture medium 43, and the detection device 100 operates in the imaging mode in which the mark 110 is most clearly detected among the plurality of imaging modes. This configuration allows the optimal imaging mode to be determined at higher accuracy because the degree of clarity of the image data is compared by actually imaging the mark 110.

### Modification

The following describes a modification of the embodiment. A detection device 100A according to the modification differs from the detection device 100 according to the embodiment in that the second module MDL2 is not provided and a third module MDL3 is provided. The imaging modes are two imaging modes, that is, the first imaging mode MOD1 and the third imaging mode MOD3. The following description focuses on differences from the embodiment.

### Configuration of Detection Device

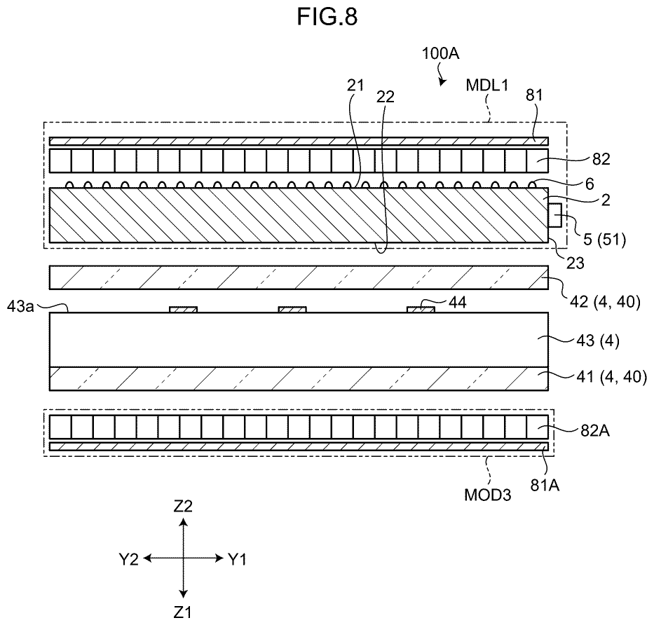

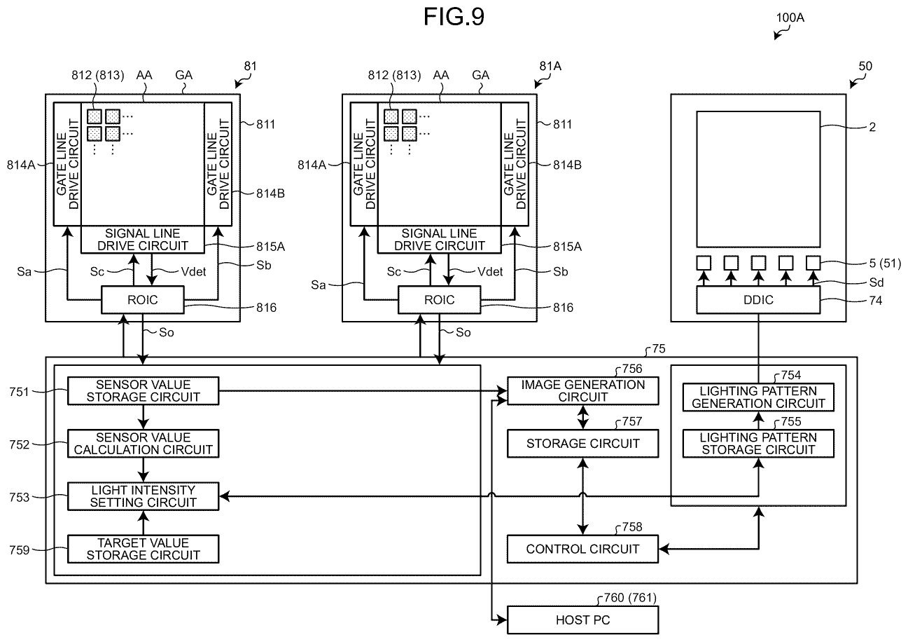

FIG. 8 is a schematic view illustrating a section of the detection device according to the modification. FIG. 9 is a block diagram illustrating a configuration example of the detection device according to the modification.

As illustrated in FIGS. 8 and 9, the detection device 100A includes the first module MDL1, the object placement member 4, and the third module MDL3. The first module MDL1, the object placement member 4, and the third module MDL3 are arranged in this order from the Z2 side (other side in the first direction) to the Z1 side (one side in the first direction).

The third module MDL3 includes the second optical sensor 81A and the second viewing angle control film 82A.

### Imaging Modes

Two imaging modes are provided. The imaging modes are the first and the third imaging modes described in the embodiment.

TABLE 3

| IMAGING MODE | MDL1 | MDL2 |
|---|---|---|
| MOD1 (REFLECTION) | FL TYPE SENSOR | NOT USED |
| MOD3 (TRANSMISSION) | LIGHT SOURCES | SENSOR |

As illustrated in TABLE 3, in the first imaging mode MOD1, the first module MDL1 is operated and the third module MDL3 is not operated. Specifically, when the first light sources 5 in the first module MDL1 are lit, the light L propagating in the first light guide plate 2 hits the first scatterers 6, is scattered, and travels toward the Z1 side. The light L is then reflected on the culture medium 43, passes through the first light guide plate 2 and the first viewing angle control film 82, and irradiates the photodiodes 813 of the first optical sensor 81.

As illustrated in TABLE 3, in the third imaging mode MOD3, the first module MDL1 and the third module MDL3 are operated. Specifically, when the first light sources 5 in the first module MDL1 are lit, the light L propagating in the first light guide plate 2 hits the first scatterers 6, is scattered, and travels toward the Z1 side. The light L then passes through the culture medium 43 and the second viewing angle control film 82A, and irradiates the photodiodes 813 of the second optical sensor 81A.

### Methods for Selecting and Determining Imaging Mode

Two methods can be used for selecting and determining the imaging mode. A second method for selection and determination will be described in detail.

### Second Method for Selection and Determination

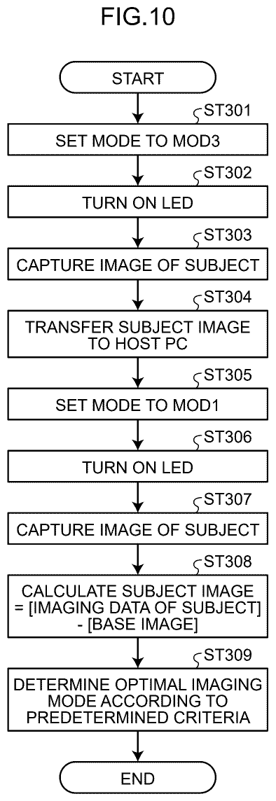

In the second method for selection and determination, a mark is attached to the culture medium, the mark is imaged (image-captured) in each of the two imaging modes, and the imaging mode that produces the clearest image of the mark is selected. FIG. 10 is a flowchart illustrating an exemplary detection operation of the detection device to select and determine the optimal imaging mode using the imaging data of the mark.

In FIG. 10, the mark 110 is imaged in the order of the third imaging mode MOD3 and the first imaging mode MOD1, and the imaging mode that produces the clearest image is determined.

First, the host IC 75 sets the imaging mode to the third imaging mode MOD3 described with reference to FIG. 3 (Step ST301). That is, the lighting pattern generation circuit 754 turns on the first light sources 5 (Step ST302) and images (captures an image of) the mark 110 serving as the subject (Step ST303). The image generation circuit 756 then generates an image of the mark 110. The image generation circuit 756 transfers the image to the host PC 760, and the host PC 760 stores therein the image (Step ST304).

Then, the host IC 75 sets the imaging mode to the first imaging mode MOD1 (Step ST305). That is, the lighting pattern generation circuit 754 turns on the first light sources 5 (Step ST306) and images (captures an image of) the mark 110 serving as the subject (Step ST307). The image generation circuit 756 then generates an image of the mark 110.

In detail, the base image is stored in advance in the generation circuit 756 performs an operation to subtract the base image from the imaging data of the mark 110 to obtain the image of the mark 110, and transfers the image of the mark 110 to the host PC 760. The host PC 760 stores therein the image (Step ST308).

Then, at Step ST309, of the two images of the mark 110, a mode that produces an image that has the largest peak gradation difference D is determined by the host IC 75 as the optimal imaging mode.

### Detection of Bacterial Colonies

Then, in the same way as in the embodiment, the culture medium 43 is imaged in the selected imaging mode to detect the development of the microorganisms such as the bacterial colonies (objects to be detected 44).

As described above, the detection device 100A according to the modification includes the first optical sensor 81, the first light guide plate 2, the object placement member 4, the second optical sensor 81A, the first light sources 5, and the first scatterers 6. The photodetection elements are the sensor pixels 812 or the photodiodes 813.

In also the modification, an imaging mode that can produce the clearest image is selected from among the plurality of imaging modes, and the objects to be detected 44 and the object placement member 4 can be imaged in the selected imaging mode. Thus, the detection device 100A that improves the accuracy of detection can be provided.

The multiple (two) imaging modes include the first imaging mode MOD1 in which the first light sources are lit and the light thereof is detected by the first optical sensor, and the third imaging mode MOD3 in which the first light sources are lit and the light thereof is detected by the second optical sensor.

Since the detection device 100A can image (that is, capture the image of) the object to be detected placement member 4 and the objects to be detected 44 using the two imaging modes, the objects to be detected 44 and the object placement member 4 can be imaged in the imaging mode that produces the clearest image among the two imaging modes.

What is claimed is:

1. A detection device comprising:
a first optical sensor comprising a plurality of photodetection elements arranged in a planar configuration;
a first light guide plate that is located on one side in a first direction with respect to the first optical sensor and has a light-transmitting property;
an object placement member that is located on the one side in the first direction with respect to the first light guide plate and on which an object to be detected is to be placed;
a second optical sensor that is located on the one side in the first direction with respect to the object placement member and comprises a plurality of photodetection elements arranged in a planar configuration; and
a first light source that is located adjacent to the first light guide plate in a second direction intersecting the first direction and is configured to emit light to a side surface of the first light guide plate, wherein
a surface on another side in the first direction of the first light guide plate is provided with a plurality of first scatterers by which light propagating in the first light guide plate reaching thereto is scattered.

2. The detection device according to claim 1, further comprising:
a second light guide plate that is located between the object placement member and the second optical sensor and has a light-transmitting property; and
a second light source that is located adjacent to the second light guide plate in the second direction and is configured to emit light to a side surface of the second light guide plate, wherein
a surface on one side in the first direction of the second light guide plate is provided with a plurality of second scatterers by which light propagating in the second light guide plate reaching thereto is scattered.

3. The detection device according to claim 2, configured to operate in any one of a plurality of imaging modes, wherein
the imaging modes comprise:
a first imaging mode in which the first light source is lit and the first optical sensor detects light of the first light source;
a second imaging mode in which the second light source is lit and the second optical sensor detects light of the second light source;
a third imaging mode in which the first light source is lit and the second optical sensor detects the light of the first light source; and
a fourth imaging mode in which the second light source is lit and the first optical sensor detects the light of the second light source.

4. The detection device according to claim 1, configured to operate in any one of a plurality of imaging modes, wherein
the imaging modes comprise:
a first imaging mode in which the first light source is lit and the first optical sensor detects light of the first light source; and
a third imaging mode in which the first light source is lit and the second optical sensor detects the light of the first light source.

5. The detection device according to claim 3, wherein
the object placement member includes a Petri dish and a culture medium accommodated in the Petri dish,
the object to be detected includes microorganisms to be provided on the culture medium,
the detection device comprises:
a storage configured to store table data indicating a relation between a combination of two or more conditions and the imaging mode;
an input device configured to receive input of a specified combination of the two or more conditions; and
a controller configured to determine the imaging mode corresponding to the combination of the conditions specified by the input with reference to the table data, and
the two or more conditions include a condition regarding transmittance of the culture medium and an orientation of a surface of the culture medium.

6. The detection device according to claim 5, wherein the condition regarding transmittance of the culture medium includes at least one of a type of the Petri dish, a thickness of the culture medium, and the transmittance of the culture medium.

7. The detection device according to claim 3, wherein
the object placement member includes a Petri dish and a culture medium accommodated in the Petri dish, and a mark is provided in advance on a part of the culture medium, and
the detection device is configured to operate in an imaging mode in which the mark is most clearly detected among the plurality of imaging modes.

8. The detection device according to claim 4, wherein
the object placement member includes a Petri dish and a culture medium accommodated in the Petri dish,
the object to be detected includes microorganisms to be provided on the culture medium,
the detection device comprises:
a storage configured to store table data indicating a relation between a combination of two or more conditions and the imaging mode;
an input device configured to receive input of a specified combination of the two or more conditions; and
a controller configured to determine the imaging mode corresponding to the combination of the conditions specified by the input with reference to the table data, and
the two or more conditions include a condition regarding transmittance of the culture medium and an orientation of a surface of the culture medium.

9. The detection device according to claim 8, wherein the condition regarding transmittance of the culture medium includes at least one of a type of the Petri dish, a thickness of the culture medium, and the transmittance of the culture medium.

10. The detection device according to claim 4, wherein
the object placement member includes a Petri dish and a culture medium accommodated in the Petri dish, and a mark is provided in advance on a part of the culture medium, and
the detection device is configured to operate in an imaging mode in which the mark is most clearly detected among the plurality of imaging modes.
